(19) United States
(12) Patent Application Publication
(10) Pub. No.: US 2023/0099381 A1
(43) Pub. Date: Mar. 30, 2023

(54) COMPUTING SYSTEM AND METHOD OF DETERMINING TARGET EPITOPE ON SPECIFIC VIRUS FOR FACILITATING DESIGN OF MUTATION-TOLERABLE VACCINE

(71) Applicant: GRAPHEN, INC., New York, NY (US)

(72) Inventors: Yi-Ting CHEN, Taipei City (TW); Sing-Han HUANG, Taipei City (TW); Ching-Yung LIN, Scarsdale, NY (US); Xiang-Yu LIN, Taipei City (TW); Cheng-Tang WANG, Taipei City (TW)

(21) Appl. No.: 17/951,252

(22) Filed: Sep. 23, 2022

Related U.S. Application Data
(60) Provisional application No. 63/248,787, filed on Sep. 27, 2021.

(51) Int. Cl. G16B 15/30; G16B 20/20

(52) U.S. Cl. CPC G16B 15/30; G16B 20/20

(57) ABSTRACT

A method includes: determining a mutation frequency for each residue in a wild-type spike protein of a specific virus; for each residue in the protein, counting a total number of contact residues related to said each residue and P antibodies based on P entries of protein structure data; for each residue in the protein, for a condition that said each residue mutates into one common amino acid residue, determining a normalized binding free energy value using a pre-established model based on the protein structure data, and determining a mutation effect score based on the mutation frequency, the total number of contact residues and the normalized binding free energy value; generating a mutation effect epitope map related to the mutation effect scores determined for all residues in the protein and all common amino acid residues; and determining, based on the map, a region in the protein as a target epitope.

### CROSS-REFERENCE TO RELATED APPLICATION

This application claims the benefit of U.S. Provisional Patent Application No. 63/248,787, filed on Sep. 27, 2021, which is incorporated by reference herein in its entirety.

### FIELD

The disclosure relates to a computing system and a method of determining a target epitope on a specific virus for facilitating design of mutation-tolerable vaccine.

### BACKGROUND

Mutations in viruses, including severe acute respiratory syndrome coronavirus 2 causing the COVID-19 pandemic (hereinafter referred to as SARS-CoV-2), may allow viruses to evade the human immune system and treatment (e.g., use of vaccines) against the viruses. For example, notable variants of SARS-CoV-2 defined by the World Health Organization (WHO), such as the United Kingdom's alpha (B.1.1.7), South Africa's beta (B.1.351), California's epsilon (B.1.429), Brazil's gamma (P.1), and India's delta (B.1.617.2), all include a spike protein where key mutations (e.g., K417N/T, L452R, T478K, E484K/Q, and N501Y) have occurred at a receptor binding domain (RBD) of the spike protein, and such mutations make these variants relatively more infectious to humans. Moreover, some mutations occur at an antigenic-supersite of an N-terminal domain (NTD) or an angiotensin-converting enzyme 2 (ACE2) binding site, which is a major target of potent virus-neutralizing antibodies against the spike protein, and thereby may reduce effectiveness of vaccines that are currently in use. Consequently, an approach to facilitate design of a mutation-tolerable vaccine is necessary.

### SUMMARY

Therefore, an object of the disclosure is to provide a computing system and a method of determining a target epitope on a specific virus for facilitating design of mutation-tolerable vaccine. The specific virus includes a wild-type coronavirus spike protein having a plurality of residues.

According to one aspect of the disclosure, the computing system includes a storage device, an input module, and a processor that is electrically connected to the storage device and the input module.

The storage device is configured to store a pre-established model.

The input module is configured to receive sequence data of a plurality of strains of the specific virus, and to receive P number of entries of protein structure data that are respectively related to P number of coronavirus spike protein-antibody complexes. Each of the P number of coronavirus spike protein-antibody complexes includes the wild-type coronavirus spike protein and a corresponding one of P number of antibodies. P is a positive integer greater than one.

The processor is configured to, for an i^{th} one of the residues in the wild-type coronavirus spike protein, determine, based on the sequence data, a mutation frequency

$${F}_{i , j} = \frac{{M}_{i , j}}{N} ,$$

where i is a positive integer ranging from one to a total number of the residues of the wild-type coronavirus spike protein, j is a positive integer ranging from one to a total number of common amino acid residues other than the i^{th} one of the residues, N represents a total number of the strains of the specific virus, and M_{i,j} represents a total number of those of the strains of the specific virus in each of which the i^{th} one of the residues has mutated into a i^{th} one of the common amino acid residues.

The processor is configured to analyze the P number of entries of protein structure data to obtain a plurality of interatomic distances respectively between a plurality of heavy-atom pairs. Each of the heavy-atom pairs includes two heavy atoms that respectively belong to the wild-type coronavirus spike protein and one of the P number of antibodies.

The processor is configured to identify, based on the interatomic distances thus obtained, all contact residues in the P number of coronavirus spike protein-antibody complexes, wherein each of the contact residues is one of the residues in the wild-type coronavirus spike protein that includes an α-carbon which is spaced apart by a distance less than 5 Å from another α-carbon of a residue of one of the P number of antibodies that is paired with the contact residue.

The processor is configured to, for each residue in the wild-type coronavirus spike protein, count a total number of the contact residues Cᵢ that are related to said each residue and the P number of antibodies.

The processor is configured to, based on the P number of entries of protein structure data, for each target interface of each residue in the wild-type coronavirus spike protein, estimate a candidate binding free energy value of the target interface by using the pre-established model. Each target interface is an interface between a mutant residue that is determined based on information related to properties of side-chain dihedral angles and bond rotation of amino acids, that possibly results from mutation of said each residue of the wild-type coronavirus spike protein and that is one of the common amino acid residues other than said each residue, and a paired residue of an antibody of one of the P number of coronavirus spike protein-antibody complexes that is paired with said each residue of the wild-type coronavirus spike protein.

The processor is configured to, for a condition that the i^{th} one of the residues in the wild-type coronavirus spike protein mutates into the j^{th} one of the common amino acid residues, select a greatest one of P number of candidate binding free energy values that are respectively related to the P number of coronavirus spike protein-antibody complexes as a representative binding free energy value B_{i,j}.

The processor is configured to, for each of the representative binding free energy values, normalize the representative binding free energy value B_{i,j} into a normalized binding free energy value H_{i,j} by using min-max scaling in a manner that the normalized binding free energy value H_{i,j} ranges from zero to one.

The processor is configured to, for the condition that the i^{th} one of the residues in the wild-type coronavirus spike protein mutates into the j^{th} one of the common amino acid residues, determine a mutation effect score E_{i,j} based on the mutation frequency F_{i,j}, the total number of the contact residues Cᵢ and the normalized binding free energy value H_{i,j} in a manner that the mutation effect score E_{i,j} ranges from zero to one.

The processor is configured to generate a mutation effect epitope map that is related to the mutation effect scores determined for all residues in the wild-type coronavirus spike protein and all of the common amino acid residues, and to determine, based on the mutation effect epitope map, a region in the wild-type coronavirus spike protein as the target epitope.

According to another aspect of the disclosure, the method is to be implemented by the computing system that is previously described. The method includes steps of:

for an i^{th} one of the residues in the wild-type coronavirus spike protein, determining, based on sequence data of a plurality of strains of the specific virus, a mutation frequency

$${F}_{i , j} = \frac{{M}_{i , j}}{N} ,$$

where i is a positive integer ranging from one to a total number of the residues of the wild-type coronavirus spike protein, j is a positive integer ranging from one to a total number of common amino acid residues other than the i^{th} one of the residues, N represents a total number of the strains of the specific virus, and M_{i,j} represents a total number of those of the strains of the specific virus in each of which the i^{th} one of the residues has mutated into a j^{th} one of the common amino acid residues;

analyzing P number of entries of protein structure data that are respectively related to P number of coronavirus spike protein-antibody complexes, each of which includes the wild-type coronavirus spike protein and a corresponding one of P number of antibodies, to obtain a plurality of interatomic distances respectively between a plurality of heavy-atom pairs, each of the heavy-atom pairs including two heavy atoms that respectively belong to the wild-type coronavirus spike protein and one of the P number of antibodies, P being a positive integer greater than one;

identifying, based on the interatomic distances, all contact residues in the P number of coronavirus spike protein-antibody complexes, wherein each of the contact residues is one of the residues in the wild-type coronavirus spike protein that includes an α-carbon which is spaced apart by a distance less than 5 Å from another α-carbon of a residue of one of the P number of antibodies that is paired with the contact residue;

for each residue in the wild-type coronavirus spike protein, counting a total number of the contact residues Cᵢ that are related to said each residue and the P number of antibodies;

based on the P number of entries of protein structure data, for each target interface of each residue in the wild-type coronavirus spike protein, estimating a candidate binding free energy value of the target interface by using a pre-established model, each target interface being an interface between a mutant residue that is determined based on information related to properties of side-chain dihedral angles and bond rotation of amino acids, that possibly results from mutation of said each residue of the wild-type coronavirus spike protein and that is one of the common amino acid residues other than said each residue, and a paired residue of an antibody of one of the P number of coronavirus spike protein-antibody complexes that is paired with said each residue of the wild-type coronavirus spike protein;

for a condition that the i^{th} one of the residues in the wild-type coronavirus spike protein mutates into the j^{th} one of the common amino acid residues, selecting a greatest one of P number of candidate binding free energy values that are respectively related to the P number of coronavirus spike protein-antibody complexes as a representative binding free energy value B_{i,j};

for each of the representative binding free energy values, normalizing the representative binding free energy value B_{i,j} into a normalized binding free energy value H_{i,j} by using min-max scaling in a manner that the normalized binding free energy value H_{i,j} ranges from zero to one;

for the condition that the i^{th} one of the residues in the wild-type coronavirus spike protein mutates into the j^{th} one of the common amino acid residues, determining a mutation effect score E_{i,j} based on the mutation frequency F_{i,j}, the total number of the contact residues Cᵢ and the normalized binding free energy value H_{i,j} in a manner that the mutation effect score E_{i,j} ranges from zero to one;

generating a mutation effect epitope map that is related to the mutation effect scores determined for all residues in the wild-type coronavirus spike protein and all of the common amino acid residues; and

determining, based on the mutation effect epitope map, a region in the wild-type coronavirus spike protein as the target epitope.

### BRIEF DESCRIPTION OF THE DRAWINGS

Other features and advantages of the disclosure will become apparent in the following detailed description of the embodiment(s) with reference to the accompanying drawings. It is noted that various features may not be drawn to scale.

FIG. 1 is a block diagram illustrating an example of a computing system for determining a target epitope on a specific virus so as to facilitate design of mutation-tolerable vaccine according to an embodiment of the disclosure.

FIG. 2 is a schematic diagram illustrating an amino acid structure.

FIG. 3 is a schematic diagram illustrating an example of a pre-established model used by the computing system for estimating a binding free energy value according to an embodiment of the disclosure.

FIG. 4 is a flow chart illustrating a method of determining a target epitope according to an embodiment of the disclosure.

FIG. 5 illustrates an exemplary plot showing total numbers of contact residues for all residues in a wild-type coronavirus spike protein.

FIG. 6 is a flow chart illustrating a method for estimating candidate binding free energy value according to an embodiment of the disclosure.

FIG. 7 is a schematic diagram illustrating an example of a mutation effect epitope map generated by the computing system according to an embodiment of the disclosure.

### DETAILED DESCRIPTION

Before the disclosure is described in greater detail, it should be noted that where considered appropriate, reference numerals or terminal portions of reference numerals have been repeated among the figures to indicate corresponding or analogous elements, which may optionally have similar characteristics.

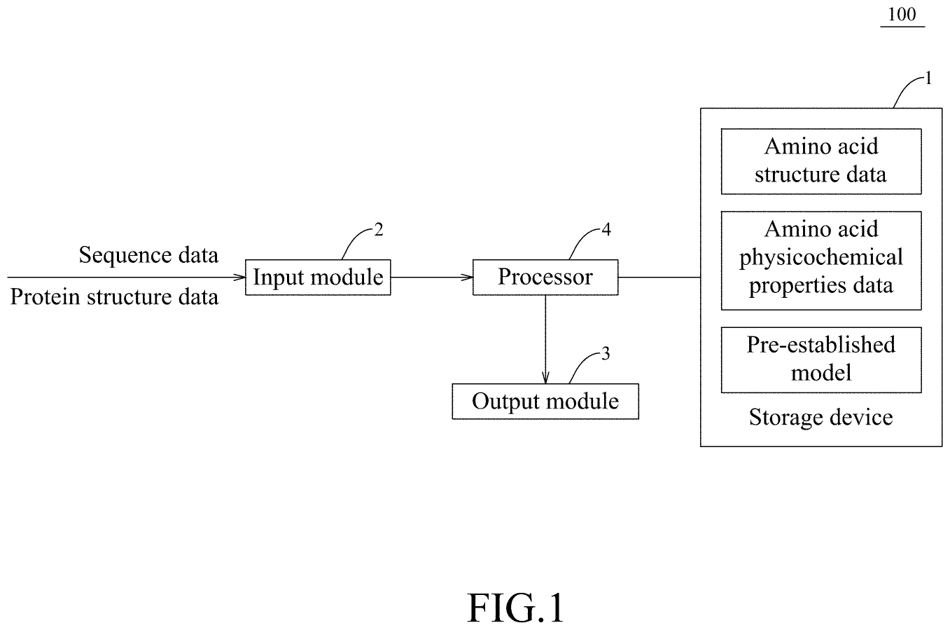

Referring to FIG. 1, an embodiment of a computing system 100 according to the disclosure is illustrated. The computer system 100 is utilized for determining a target epitope on a specific virus so as to facilitate design of mutation-tolerable vaccine. In this embodiment, the specific virus is exemplarily severe acute respiratory syndrome coronavirus 2 (SARS-CoV-2), but is not limited thereto. The specific virus includes a wild-type coronavirus spike protein having a plurality of residues.

The computing system 100 may be implemented to be a desktop computer, a laptop computer, a notebook computer or a tablet computer, but implementation thereof is not limited to what are disclosed herein and may vary in other embodiments. The computer system 100 includes a storage device 1, an input module 2, an output module 3, and a processor 4 that is electrically connected to the storage device 1, the input module 2 and the output module 3.

The storage device 1 may be implemented by random access memory (RAM), double data rate synchronous dynamic random access memory (DDR SDRAM), read only memory (ROM), programmable ROM (PROM), flash memory, one or more hard disk drives (HDDs), one or more solid state disks (SSDs), electrically-erasable programmable read-only memory (EEPROM) or any other volatile/non-volatile memory devices, but is not limited thereto. The storage device 1 is configured to store amino acid structure data, amino acid physicochemical properties data and a pre-established model.

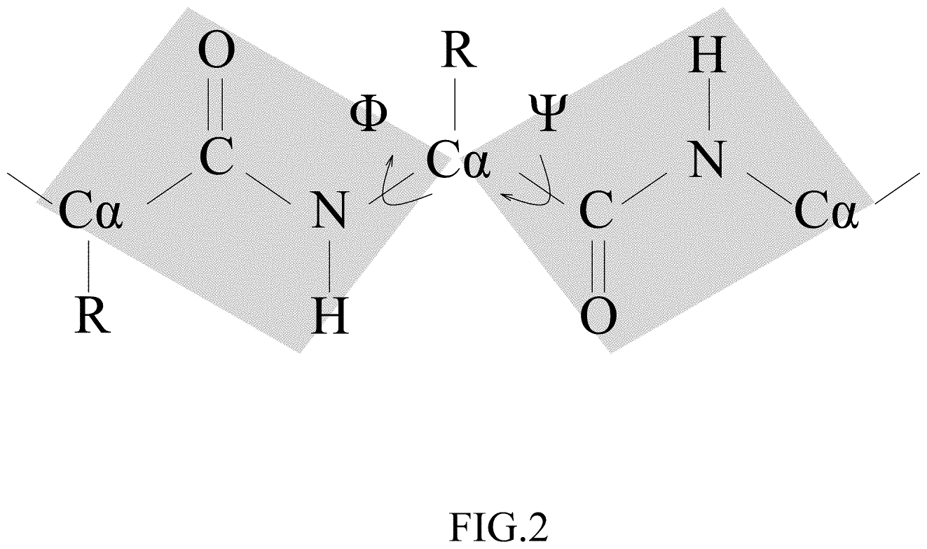

The amino acid structure data contains information related to properties of backbone dihedral angles, side-chain dihedral angles and bond rotation of amino acids. It is worth to note that in regard to amino acids of a protein (see FIG. 2), two bonds “Cα-N” and “Cα-C” that are respectively at two sides of an α-carbon (Cα) are each freely rotatable. In addition, chains “Cα-N—C—Cα” and “Cα-C—N—Cα” at two sides of the α-carbons (Cα) respectively define two planes (which are colored in grey in FIG. 2). An internal angle between two intersecting planes defined by chain “C—N—Cα-C” is referred to as a backbone dihedral angle “Φ”, an internal angle between two intersecting planes defined by chain “N—Cα-C—N” is referred to as a backbone dihedral angle “Ψ”, and an internal angle between two intersecting planes defined by chain “N—Cα-Cβ-XG” (not shown) is referred to as a sidechain dihedral angle “Xₙ” (where n is an integer such as one). Since properties of backbone dihedral angles, side-chain dihedral angles and bond rotation of amino acids are well known to one skilled in the relevant art, detailed explanation of the same is omitted herein for the sake of brevity.

The amino acid physicochemical properties data contains information related to physicochemical properties of at least 20 amino acids (hereinafter also referred to as common amino acids), including alanine (i.e., Ala or A), arginine (i.e., Arg or R), asparagine (i.e., Asn or N), aspartate (i.e., Asp or D), cysteine (i.e., Cys or C), glutamine (i.e., Gln or Q), glutamate (i.e., Glu or E), glycine (i.e., Gly or G), histidine (i.e., His or H), isoleucine (i.e., Ile or I), leucine (i.e., Leu or L), lysine (i.e., Lys or K), methionine (i.e., Met or M), phenylalanine (i.e., Phe or F), proline (i.e., Pro or P), serine (i.e., Ser or S), threonine (i.e., Thr or T), tryptophan (i.e., Trp or W), tyrosine (i.e., Tyr or Y), and valine (i.e., Val or V), but are not limited to what are disclosed herein. Based on physicochemical properties of side chains of amino acids, the amino acids can be exemplarily classified into amino acids with positively or negatively charged side chains, amino acids with polar side chains, amino acids with hydrophobic side chains, and amino acids with special side chains. Physicochemical properties of amino acids can be exemplarily encoded by five bits of binary digits, wherein for the five bits from left to right, a first bit being “1” indicates an amino acid with a positively charged side chain, a second bit being “1” indicates an amino acid with a negatively charged side chain, a third bit being “1” indicates an amino acid with a polar side chain, a fourth bit being “1” indicates an amino acid with a hydrophobic side chain, and a fifth bit being “1” indicates an amino acid with a special side chain. For example, physicochemical properties of asparagine (N), which is an amino acid with a polar side chain, would be encoded by binary digits “00100”. Since physicochemical properties of amino acids are well known to one skilled in the relevant art, detailed explanation of the same is omitted herein for the sake of brevity.

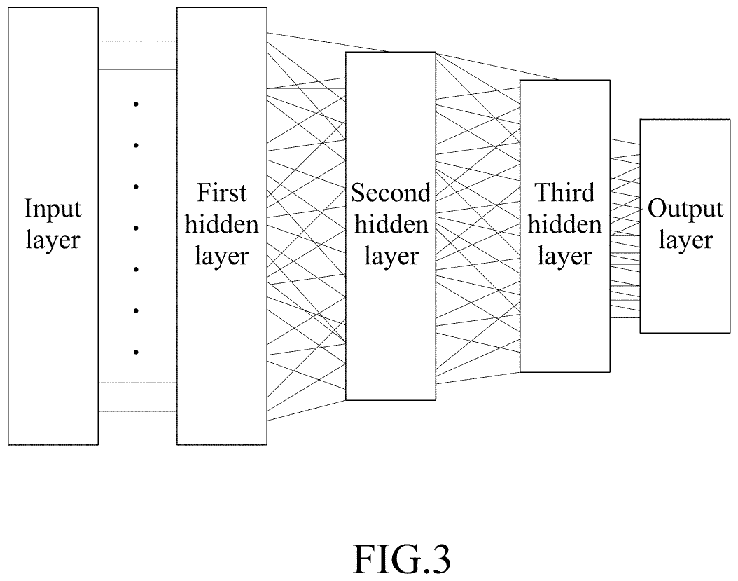

The pre-established model is implemented by a deep neural network (DNN). Referring to FIG. 3, in this embodiment, the pre-established model includes an input layer, three hidden layers and an output layer. For example, a first one of the three hidden layers (also referred to as a first hidden layer) includes 64 neurons and is implemented by a rectified linear unit (ReLU) activation function, a second one of the three hidden layers (also referred to as a second hidden layer) includes 32 neurons and is also implemented by the ReLU activation function, and a third one of the three hidden layers (also referred to as a third hidden layer) includes 16 neurons and is also implemented by the ReLU activation function.

The pre-established model is trained in advance by using a plurality of training sets that respectively correspond to a plurality of training protein complexes. Each of the training protein complexes includes at least one pair of training residues that are respectively in two protein chains of the training protein complex and that are related to a training interaction interface. Each of the training sets contains, for each pair of training residues included in the corresponding one of the training protein complexes, an atomic distance that is related to the training interaction interface, an atomic interaction force of the training interaction interface, binding free energy value of the training interaction interface, and information related to physicochemical properties of amino acids that are related to the pair of training residues. The input layer of the pre-established model is configured to receive the atomic distance(s), the atomic interaction force(s) and the information that are contained in each of the training sets and that are fed into the pre-established model, and the output layer of the pre-established model is configured to output binding free energy value(s) estimated by the pre-established model.

In one embodiment, the input module 2 is embodied using a network interface controller or a wireless transceiver that supports wireless communication standards, such as Bluetooth® technology standards, Wi-Fi technology standards and/or cellular network technology standards. The input module 2 is connected to a telecommunications network (not shown) for receiving data transmitted by a remote device (e.g., a data server).

In one embodiment, the input module 2 is embodied using a keyboard, a mouse, and/or a touch panel that is configured to present a graphical user interface. However, it should be noted that implementations of the input module 2 are not limited to what are disclosed herein and may vary in other embodiments.

In this embodiment, the input module 2 is configured to receive, from a database of Global Initiative on Sharing Avian Influenza Data (GISAID), sequence data of about two millions (e.g., 1,938,659) strains of the specific virus, and to receive, from a database of Protein Data Bank (PDB), P number of entries of protein structure data that are respectively related to P number of coronavirus spike protein-antibody complexes, where P is a positive integer greater than one, e.g., 145. Each of the P number of coronavirus spike protein-antibody complexes includes the wild-type coronavirus spike protein and a corresponding one of P number of antibodies. Each of the P number of entries of protein structure data contains spatial coordinate sets respectively of all atoms of the respective one of the P number of coronavirus spike protein-antibody complexes. Each of the spatial coordinate sets may be represented by a 3-tuple in a Cartesian coordinate system, but is not limited thereto.

The output module 3 may be embodied using a display device (e.g., a liquid-crystal display (LCD), a light-emitting diode (LED) display, a plasma display panel, a projection display or the like). However, implementation of the output module 3 is not limited to the disclosure herein and may vary in other embodiments.

The processor 4 may be implemented by a central processing unit (CPU), a microprocessor, a micro control unit (MCU), a system on a chip (SoC), or any circuit configurable/programmable in a software manner and/or hardware manner to implement functionalities discussed in this disclosure.

For an i^{th} one of the residues in the wild-type coronavirus spike protein, the processor 4 is configured to determine, based on the sequence data received by the input module 2, a mutation frequency

$${F}_{i , j} = \frac{{M}_{i , j}}{N} \times 100 \% ,$$

where i is a positive integer ranging from one to a total number of the residues of the wild-type coronavirus spike protein, j is a positive integer ranging from one to a total number of common amino acid residues other than the i^{th} one of the residues, N represents a total number of the strains of the specific virus, and Mᵢ represents a total number of those of the strains of the specific virus in each of which the i^{th} one of the residues has mutated into a j^{th} one of the common amino acid residues. For example, the total number of the residues of the wild-type coronavirus spike protein is 1267, the total number of common amino acid residues is 20, and the total number of the strains of the specific virus is 1,938,659. In other words, i=1, 2, . . . , 1267, j=1, 2, . . . , 19, and N=1938659. It is worth to note that each of mutation frequencies respectively corresponding to residues N501, D614, P681 and D1118 in the wild-type coronavirus spike protein exceeds 40%. In particular, the mutation frequency corresponding to the residue D614 is close to 100%.

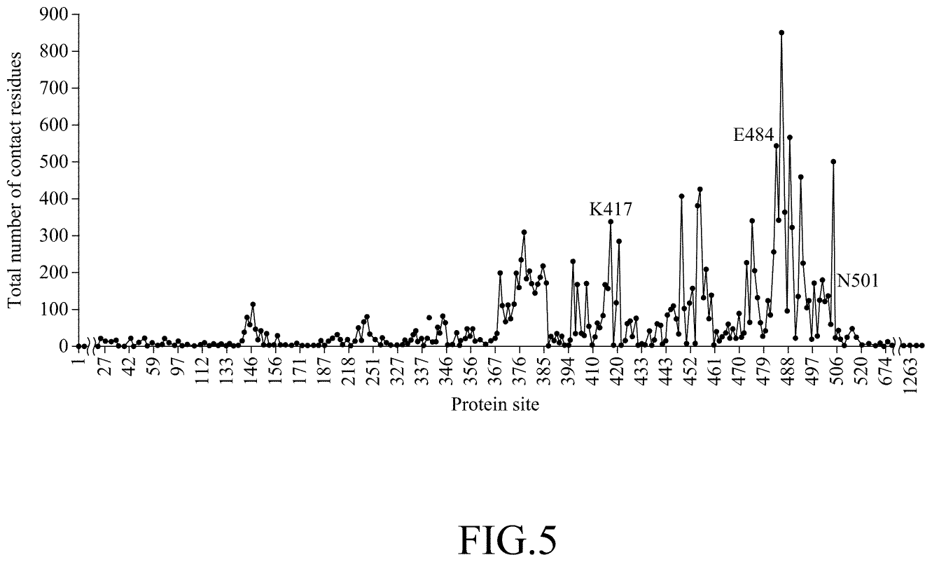

The processor 4 is configured to analyze the P number of entries of protein structure data to obtain a plurality of interatomic distances respectively between a plurality of heavy-atom pairs. Each of the heavy-atom pairs includes two heavy atoms that respectively belong to the wild-type coronavirus spike protein and one of the P number of antibodies. A heavy atom is an atom other than hydrogen, such as oxygen, nitrogen or carbon. The processor 4 is further configured to identify, based on the interatomic distances thus obtained, all contact residues in the P number of coronavirus spike protein-antibody complexes, wherein each of the contact residues is one of the residues in the wild-type coronavirus spike protein that includes an α-carbon which is spaced apart by a distance less than 5 Å from another α-carbon of a residue of one of the P number of antibodies paired with said contact residue. For each residue in the wild-type coronavirus spike protein, the processor 4 is further configured to count a total number of the contact residues Cᵢ that are related to said each residue and the P number of antibodies. For the i^{th} one of the residues in the wild-type coronavirus spike protein, the total number of the contact residues Cᵢ can be expressed as Cᵢ=Σk₌₁^{p}C_{i,k}, where C_{i,k} represents a number of times when the i^{th} one of the residues in the wild-type coronavirus spike protein is identified as the contact residue with respect to a k^{th} one of the P number of antibodies, and k is a positive integer ranging from 1 to P. FIG. 5 illustrates an example of a plot showing the total numbers of the contact residues for all residues in the wild-type coronavirus spike protein. The total number of the contact residues C₄₈₄ corresponding to a residue E484 in the wild-type coronavirus spike protein is equal to 543, which means that the residue E484 of the specific virus is prone to having interaction with antibodies.

Based on the P number of entries of protein structure data, for each target interface of each residue in the wild-type coronavirus spike protein, the processor 4 is configured to estimate a candidate binding free energy value of the target interface by using the pre-established model. For each residue in the wild-type coronavirus spike protein, each target interface is an interface between (a) a (possible) mutant residue that is determined based on information related to properties of side-chain dihedral angles and bond rotation of amino acids, that possibly results from mutation of said each residue of the wild-type coronavirus spike protein and that is one of the common amino acid residues other than said each residue, and (b) a residue of an antibody of one of the P number of coronavirus spike protein-antibody complexes that is paired with said mutant residue (hereinafter referred to as “paired residue” of the antibody). It should be noted herein that a residue of the wild-type coronavirus spike protein might possibly mutate into one of several (e.g., X number of) different mutant residues, and for each residue of the wild-type coronavirus spike protein, the processor 4 is configured to estimate, with respect to each of the P number of antibodies, multiple (X number of) candidate binding free energy values that respectively correspond to multiple (X number of) target interfaces, each of which is between a possible mutant residue into which said residue of the wild-type coronavirus spike protein might possibly mutate and a residue of the antibody; as such, X times P number of candidate binding free energy values will be estimated with respect to each residue of the wild-type coronavirus spike protein. For ease of illustration, in the following description, it will be assumed that each residue of the wild-type coronavirus spike protein corresponds to only one mutant residue.

Specifically, for each of the P number of entries of protein structure data, the processor 4 is configured to obtain, from the entry of protein structure data, the spatial coordinate sets respectively of all heavy atoms of the corresponding one of the P number of coronavirus spike protein-antibody complexes; and for each residue in the wild-type coronavirus spike protein of one of the P number of coronavirus spike protein-antibody complexes that corresponds to said entry of protein structure data, the processor 4 is configured to determine, based on information related to properties of side-chain dihedral angles and bond rotation of amino acids, a mutant residue that possibly results from mutation of said each residue of the wild-type coronavirus spike protein. The processor 4 is configured to obtain, from the amino acid structure data, an inferred rotation angle that is related to a side chain of said each residue of the wild-type coronavirus spike protein, and to calculate spatial coordinate sets respectively of all heavy atoms of the mutant residue based on the spatial coordinate sets of all heavy atoms of said each residue of the wild-type coronavirus spike protein and the inferred rotation angle.

For each of the P number of entries of protein structure data, for each residue in the wild-type coronavirus spike protein, for a condition that said each residue mutates into the mutant residue, and for a target interface between the mutant residue and a paired residue of the corresponding one of P number of antibodies, the processor 4 is further configured to perform the following operations. For every two heavy atoms respectively of the mutant residue and the paired residue of the corresponding one of P number of antibodies (hereinafter referred to as “a mutant-residue-paired-residue heavy atom pair” of a residue complex, wherein the residue complex corresponds to said each mutated residue in the wild-type coronavirus spike protein and the corresponding one of the P number of antibodies), the processor 4 is configured to calculate a value of atomic-level energy and an Euclidean distance based on the spatial coordinate sets of the heavy atoms of the corresponding one of the P number of coronavirus spike protein-antibody complexes and the spatial coordinate sets of the heavy atoms of the mutant residue, and to calculate an atomic distance related to the target interface and an atomic interaction force of the target interface based on the values of atomic-level energy and the Euclidean distances thus calculated for all the mutant-residue-paired-residue heavy atom pairs of the residue complex. Then, the processor 4 is configured to obtain, from the amino acid physicochemical properties data, relevant information that is related to said each residue of the wild-type coronavirus spike protein and the corresponding mutant residue, and to estimate the candidate binding free energy value of the target interface by feeding, into the pre-established model, the atomic distance related to the target interface, the atomic interaction force of the target interface, and the relevant information.

In particular, the processor 4 is configured to calculate, for the two heavy atoms of each mutant-residue-paired-residue heavy atom pair of the residue complex, the value of atomic-level energy as a sum of values of Van der Waals force, hydrogen bond, π-π stacking interaction and electrostatic force between the two heavy atoms of the mutant-residue-paired-residue heavy atom pair. Thereafter, the processor 4 is further configured to calculate the atomic distance as an average of the Euclidean distances of all mutant-residue-paired-residue heavy atom pairs of the residue complex, and to calculate the atomic interaction force as a sum of the values of atomic-level energy of all mutant-residue-paired-residue heavy atom pairs of the residue complex. Mathematically, the atomic distance (D) and the atomic interaction force (E) can be respectively expressed by

$$D = \frac{{\sum }_{t = 1}^{Q} {d}_{t}}{Q} , and E = {\sum }_{t = 1}^{Q} {e}_{t} ,$$

where Q is a total number of the mutant-residue-paired-residue heavy atom pairs of the residue complex, dₜ represents an Euclidean distance of a t^{th} one of the mutant-residue-paired-residue heavy atom pairs of the residue complex, and eₜ represents an atomic-level energy of the t^{th} one of the mutant-residue-paired-residue heavy atom pairs of the residue complex. Since calculations of Van der Waals force, hydrogen bond, π-π stacking interaction and electrostatic force are well known to one skilled in the relevant art, detailed explanation of the same is omitted herein for the sake of brevity.

It should be noted that since implementation of estimating binding free energy value has been disclosed in U.S. patent application Ser. No. 17/865,140, the entire content of which is hereby incorporated by reference, detailed explanation of the same is omitted herein for the sake of brevity.

Thereafter, for the condition that the i^{th} one of the residues in the wild-type coronavirus spike protein mutates into the j^{th} one of the common amino acid residues, the processor 4 is configured to select a greatest one of P number of candidate binding free energy values that are respectively related to the P number of coronavirus spike protein-antibody complexes as a representative binding free energy value B_{i,j}. In addition, for each of the representative binding free energy values, the processor 4 is configured to normalize the representative binding free energy value B_{i,j} into a normalized binding free energy value H_{i,j} by using min-max scaling in a manner that the normalized binding free energy value H_{i,j} ranges from zero to one. Specifically, the processor 4 is configured to normalize the representative binding free energy value B_{i,j} by calculating the normalized binding free energy value as

$${H}_{i , j} = \frac{{B}_{i , j} - min ( B )}{max ( B ) - min ( B )} ,$$

where max(B) represents a greatest one of all of the representative binding free energy values (i.e., the greatest one among 1267×19 number of the representative binding free energy values), and min(B) represents a smallest one of all of the representative binding free energy values (i.e., the smallest one among the 1267×19 number of the representative binding free energy values).

For the condition that the i^{th} one of the residues in the wild-type coronavirus spike protein mutated into the j^{th} one of the common amino acid residues, the processor 4 is configured to determine a mutation effect score E_{i,j} based on the mutation frequency F_{i,j}, the total number of the contact residues Cᵢ and the normalized binding free energy value H_{i,j} in a manner that the mutation effect score E_{i,j} ranges from zero to one. Specifically, the processor 4 is configured to determine the mutation effect score E_{i,j} by calculating the mutation effect score as

$${E}_{i , j} = \frac{{F}_{i , j} + \frac{{C}_{i}}{max ( C ) - min ( C )} + {H}_{i , j}}{3} ,$$

where max(C) represents a greatest one of all of the total numbers of the contact residues (i.e., the greatest one among C₁ to C₁₂₆₇), and min(C) represents a smallest one of all of the total numbers of the contact residues (i.e., the smallest one among C₁ to C₁₂₆₇). It should be noted that the total numbers of the contact residues correspond respectively to the residues in the wild-type coronavirus spike protein.

The processor 4 is configured to generate a mutation effect epitope map that is related to the mutation effect scores determined for all residues in the wild-type coronavirus spike protein and all of the common amino acid residues (i.e., there will be 1267×19 number of the mutation effect scores), and to determine, based on the mutation effect epitope map, a region in the wild-type coronavirus spike protein as the target epitope. Then, the processor 4 is configured to control the output module 3 to present the mutation effect epitope map and the target epitope for relevant technicians (e.g., members of a research and development team in charge of developing vaccines for the specific virus).

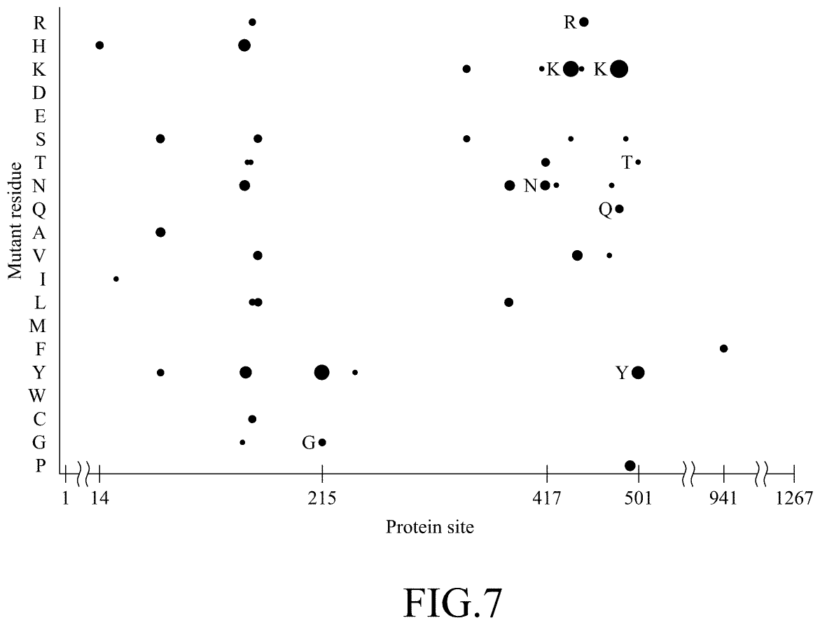

FIG. 7 illustrates an example of the mutation effect epitope map, which is generated based on the 1,938,659 strains of the specific virus and the 145 entries of protein structure data that are previously described. In the mutation effect epitope map, a size of a black dot reflects a grade of a mutation effect score. That is to say, the larger the black dot, the higher the mutation effect score. Besides, an alphabet marked beside a black dot indicates a mutant residue (i.e., one of the common amino acid residues other than a wild-type residue in the wild-type coronavirus spike protein) that has been found in the specific virus.

It should be noted that the processor 4 is configured to determine the target epitope based on the following principles. First, one region of the wild-type coronavirus spike protein having less mutant residues than other regions of the wild-type coronavirus spike protein means that a probability of mutations occurring in that region is lower than a probability of mutations occurring in each of said other regions. Second, one region of the wild-type coronavirus spike protein having mutation effect score(s) lower than those in other regions of the wild-type coronavirus spike protein means that a mutant residue in that region would have a relatively small or insignificant impact on stability of binding of a coronavirus spike protein-antibody complex. For example, according to the above-mentioned principles, the processor 4 would determine, with respect to the mutation effect epitope map shown in FIG. 7, a region on the mutation effect epitope map from 216^{th} to 416^{th} ones of the residues of the wild-type coronavirus spike protein as the target epitope. It is worth to note that since few residue mutations occur within a target epitope of an antigen, and since a mutant residue in the target epitope has relatively small impact on stability of binding of an antigen-antibody complex, vaccines designed based on the target epitope may still maintain its effectiveness even when mutations occur in the target epitope.

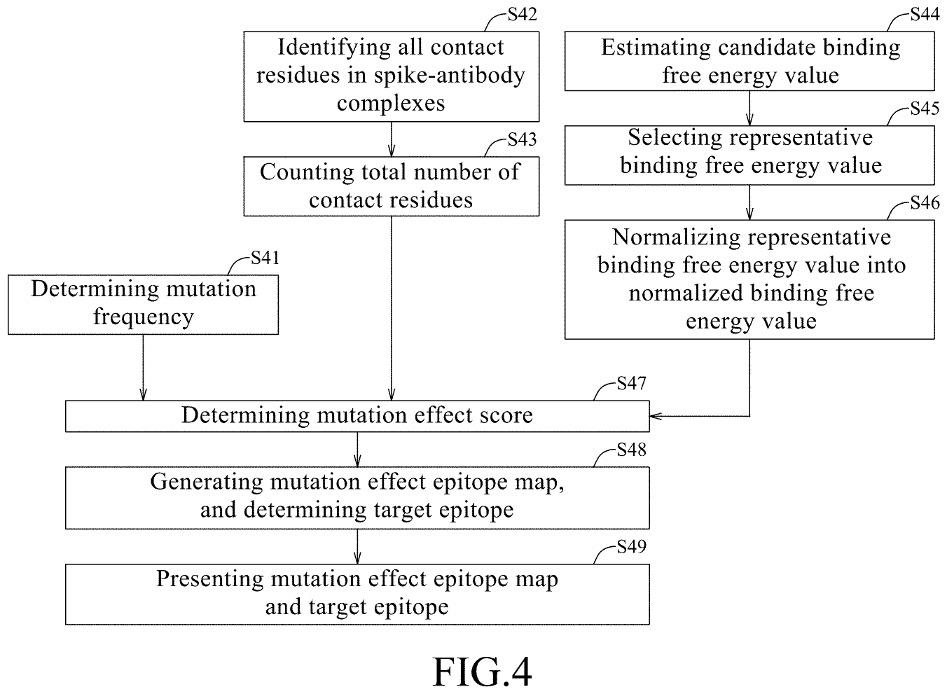

Referring to FIG. 4, an embodiment of a method of determining a target epitope according to the disclosure is illustrated. The method is adapted to be implemented by the computing system 100 that is previously described. The method includes steps S41 to S49 delineated below.

In step S41, for the i^{th} one of the residues in the wild-type coronavirus spike protein, the processor 4 determines the mutation frequency

$${F}_{i , j} = \frac{{M}_{i , j}}{N}$$

based on the sequence data.

In step S42, the processor 4 analyzes the P number of entries of protein structure data to obtain the interatomic distances respectively between the heavy-atom pairs, and identifies, based on the interatomic distances, all contact residues in the P number of coronavirus spike protein-antibody complexes.

In step S43, for each residue in the wild-type coronavirus spike protein, the processor 4 counts the total number of the contact residues Cᵢ that are related to said each residue and the P number of antibodies.

In step S44, based on the P number of entries of protein structure data, for each target interface of each residue in the wild-type coronavirus spike protein, the processor 4 estimates the candidate binding free energy value of the target interface by using the pre-established model.

In step S45, for a condition that the i^{th} one of the residues in the wild-type coronavirus spike protein mutates into the j^{th} one of the common amino acid residues, the processor 4 selects a greatest one of the P number of candidate binding free energy values as the representative binding free energy value B_{i,j}. This step is performed with respect to every value of i and every value of j.

In step S46, for each of the representative binding free energy values obtained in step S45, the processor 4 normalizes the representative binding free energy value B_{i,j} into the normalized binding free energy value H_{i,j} by using min-max scaling.

In step S47, for the condition that the i^{th} one of the residues in the wild-type coronavirus spike protein mutates into the j^{th} one of the common amino acid residues, the processor 4 determines the mutation effect score E_{i,j} based on the mutation frequency F_{i,j}, the total number of the contact residues Cᵢ and the normalized binding free energy value H_{i,j}. This step is performed with respect to every value of i and every value of j.

In step S48, the processor 4 generates the mutation effect epitope map, and determines, based on the mutation effect epitope map, a region in the wild-type coronavirus spike protein as the target epitope.

In step S49, the processor 4 controls the output module 3 to present the mutation effect epitope map and the target epitope.

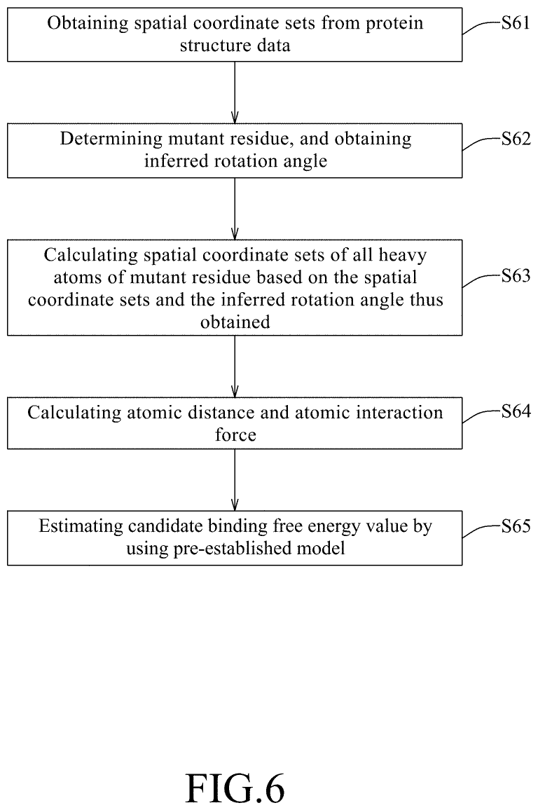

Referring to FIG. 6, an embodiment of a method for estimating a candidate binding free energy value according to the disclosure is illustrated. For each of the P number of entries of protein structure data, the processor 4 executes steps S61 to S65 delineated below.

In step S61, from the entry of protein structure data, the processor 4 obtains spatial coordinate sets respectively of all heavy atoms of the corresponding one of the P number of coronavirus spike protein-antibody complexes.

In step S62, for each residue in the wild-type coronavirus spike protein of the corresponding one of the P number of coronavirus spike protein-antibody complexes, the processor 4 determines, based on information related to properties of side-chain dihedral angles and bond rotation of amino acids, a mutant residue that possibly results from mutation of said each residue of the wild-type coronavirus spike protein. Then, the processor 4 obtains, from the amino acid structure data, an inferred rotation angle that is related to a side chain of said each residue of the wild-type coronavirus spike protein.

In step S63, for each residue in the wild-type coronavirus spike protein of the corresponding one of the P number of coronavirus spike protein-antibody complexes, the processor 4 calculates spatial coordinate sets respectively of all heavy atoms of the mutant residue based on the spatial coordinate sets of all heavy atoms of said each residue of the wild-type coronavirus spike protein and the inferred rotation angle.

In step S64, for each residue in the wild-type coronavirus spike protein of the corresponding one of the P number of coronavirus spike protein-antibody complexes, for a target interface between the mutant residue and a paired residue of the corresponding one of P number of antibodies, and for every two heavy atoms respectively of the mutant residue and the paired residue of the corresponding one of P number of antibodies, the processor 4 calculates the value of atomic-level energy and the Euclidean distance based on the spatial coordinate sets of the heavy atoms of the corresponding one of the P number of coronavirus spike protein-antibody complexes and the spatial coordinate sets of the heavy atoms of the mutant residue. Thereafter, the processor 4 calculates, based on the values of atomic-level energy and the Euclidean distances thus calculated, the atomic distance related to the target interface and the atomic interaction force of the target interface.

In step S65, for each residue in the wild-type coronavirus spike protein, the processor 4 obtains, from the amino acid physicochemical properties data, relevant information that is related to said each residue of the wild-type coronavirus spike protein and the mutant residue. Afterwards, the processor 4 estimates the candidate binding free energy value of the target interface by feeding, into the pre-established model, the atomic distance related to the target interface, the atomic interaction force of the target interface, and the relevant information.

To sum up, for the method and the computing system 100 according to the disclosure, the mutation effect epitope map, which is related to the mutation effect scores determined for all residues in the wild-type coronavirus spike protein and all of the common amino acid residues, is eventually obtained. The mutation effect scores is determined based on the mutation frequencies, the total numbers of contact residues and the normalized binding free energy values. Based on the mutation effect epitope map, a region in the wild-type coronavirus spike protein can be determined as the target epitope, which may be a potential target for design of mutation-tolerable vaccine. The method and the computing system 100 according to the disclosure may facilitate development of mutation-tolerable vaccines by directing research and development energy and efforts to more probable targets, and significantly shortening the time spent on experimentation during the search for a workable target, thereby contributing to the public health in the face of a pandemic.

In the description above, for the purposes of explanation, numerous specific details have been set forth in order to provide a thorough understanding of the embodiment(s). It will be apparent, however, to one skilled in the art, that one or more other embodiments may be practiced without some of these specific details. It should also be appreciated that reference throughout this specification to “one embodiment,” “an embodiment,” an embodiment with an indication of an ordinal number and so forth means that a particular feature, structure, or characteristic may be included in the practice of the disclosure. It should be further appreciated that in the description, various features are sometimes grouped together in a single embodiment, figure, or description thereof for the purpose of streamlining the disclosure and aiding in the understanding of various inventive aspects; such does not mean that every one of these features needs to be practiced with the presence of all the other features. In other words, in any described embodiment, when implementation of one or more features or specific details does not affect implementation of another one or more features or specific details, said one or more features may be singled out and practiced alone without said another one or more features or specific details. It should be further noted that one or more features or specific details from one embodiment may be practiced together with one or more features or specific details from another embodiment, where appropriate, in the practice of the disclosure.

While the disclosure has been described in connection with what is(are) considered the exemplary embodiment(s), it is understood that this disclosure is not limited to the disclosed embodiment(s) but is intended to cover various arrangements included within the spirit and scope of the broadest interpretation so as to encompass all such modifications and equivalent arrangements.

What is claimed is:

1. A method of determining a target epitope on a specific virus for facilitating design of mutation-tolerable vaccine, to be implemented by a computing system, the specific virus including a wild-type coronavirus spike protein having a plurality of residues, the method comprising steps of:
for an i^{th} one of the residues in the wild-type coronavirus spike protein, determining, based on sequence data of a plurality of strains of the specific virus, a mutation frequency
$${F}_{i , j} = \frac{{M}_{i , j}}{N} ,$$
where i is a positive integer ranging from one to a total number of the residues of the wild-type coronavirus spike protein, j is a positive integer ranging from one to a total number of common amino acid residues other than the i^{th} one of the residues, N represents a total number of the strains of the specific virus, and M_{i,j} represents a total number of those of the strains of the specific virus in each of which the i^{th} one of the residues has mutated into a j^{th} one of the common amino acid residues;
analyzing P number of entries of protein structure data that are respectively related to P number of coronavirus spike protein-antibody complexes, each of which includes the wild-type coronavirus spike protein and a corresponding one of P number of antibodies, to obtain a plurality of interatomic distances respectively between a plurality of heavy-atom pairs, each of the heavy-atom pairs including two heavy atoms that respectively belong to the wild-type coronavirus spike protein and one of the P number of antibodies, P being a positive integer greater than one;
identifying, based on the interatomic distances, all contact residues in the P number of coronavirus spike protein-antibody complexes, wherein each of the contact residues is one of the residues in the wild-type coronavirus spike protein that includes an α-carbon which is spaced apart by a distance less than 5 Å from another α-carbon of a residue of one of the P number of antibodies that is paired with the contact residue;
for each residue in the wild-type coronavirus spike protein, counting a total number of the contact residues Cᵢ that are related to said each residue and the P number of antibodies;
based on the P number of entries of protein structure data, for each target interface of each residue in the wild-type coronavirus spike protein, estimating a candidate binding free energy value of the target interface by using a pre-established model, each target interface being an interface between a mutant residue that is determined based on information related to properties of side-chain dihedral angles and bond rotation of amino acids, that possibly results from mutation of said each residue of the wild-type coronavirus spike protein and that is one of the common amino acid residues other than said each residue, and a paired residue of an antibody of one of the P number of coronavirus spike protein-antibody complexes that is paired with said each residue of the wild-type coronavirus spike protein;
for a condition that the i^{th} one of the residues in the wild-type coronavirus spike protein mutates into the j^{th} one of the common amino acid residues, selecting a greatest one of P number of candidate binding free energy values that are respectively related to the P number of coronavirus spike protein-antibody complexes as a representative binding free energy value B_{i,j};
for each of the representative binding free energy values, normalizing the representative binding free energy value B_{i,j} into a normalized binding free energy value H_{i,j} by using min-max scaling in a manner that the normalized binding free energy value H_{i,j} ranges from zero to one;
for the condition that the i^{th} one of the residues in the wild-type coronavirus spike protein mutates into the j^{th} one of the common amino acid residues, determining a mutation effect score E_{i,j} based on the mutation frequency F_{i,j}, the total number of the contact residues Cᵢ and the normalized binding free energy value H_{i,j} in a manner that the mutation effect score E_{i,j} ranges from zero to one;
generating a mutation effect epitope map that is related to the mutation effect scores determined for all residues in the wild-type coronavirus spike protein and all of the common amino acid residues; and
determining, based on the mutation effect epitope map, a region in the wild-type coronavirus spike protein as the target epitope.

2. The method as claimed in claim 1, further comprising a step of presenting the mutation effect epitope map and the target epitope.

3. The method as claimed in claim 1, wherein the step of normalizing the representative binding free energy value B_{i,j} is to calculate the normalized binding free energy value as
$${H}_{i , j} = \frac{{B}_{i , j} - min ( B )}{max ( B ) - min ( B )} ,$$
where max(B) represents a greatest one of all of the representative binding free energy values, and min(B) represents a smallest one of all of the representative binding free energy values.

4. The method as claimed in claim 1, wherein the step of determining a mutation effect score E_{i,j} is to calculate the mutation effect score as
$${E}_{i , j} = \frac{{F}_{i , j} + \frac{{C}_{i}}{max ( C ) - min ( C )} + {H}_{i , j}}{3} ,$$
where max(C) represents a greatest one of all of the total numbers of the contact residues, and min(C) represents a smallest one of all of the total numbers of the contact residues, the total numbers of the contact residues corresponding respectively to the residues in the wild-type coronavirus spike protein.

5. The method as claimed in claim 1, wherein the pre-established model is implemented by a deep neural network (DNN), and is trained by using a plurality of training sets that respectively correspond to a plurality of training protein complexes, each of the training protein complexes including at least one pair of training residues that are respectively in two protein chains of the training protein complex and that are related to a training interaction interface, each of the training sets contains, for each of the at least one pair of training residues included in the corresponding one of the training protein complexes, an atomic distance that is related to the training interaction interface, an atomic interaction force of the training interaction interface, a binding free energy value of the training interaction interface, and information related to physicochemical properties of amino acids that are related to the pair of training residues.

6. The method as claimed in claim 1, wherein each of the P number of entries of protein structure data contains spatial coordinate sets respectively of all atoms of the corresponding one of the P number of coronavirus spike protein-antibody complexes, for each of the P number of entries of protein structure data, the method further comprising steps of, before the step of estimating a candidate binding free energy value:
from the entry of protein structure data, obtaining spatial coordinate sets respectively of all heavy atoms of the corresponding one of the P number of coronavirus spike protein-antibody complexes;
for each residue in the wild-type coronavirus spike protein of the corresponding one of the P number of coronavirus spike protein-antibody complexes,
determining, based on information related to properties of side-chain dihedral angles and bond rotation of amino acids, a mutant residue that possibly results from mutation of said each residue of the wild-type coronavirus spike protein,
obtaining an inferred rotation angle that is related to a side chain of said each residue of the wild-type coronavirus spike protein from amino acid structure data that contains information related to properties of backbone dihedral angles, side-chain dihedral angles and bond rotation of amino acids, and
calculating spatial coordinate sets respectively of all heavy atoms of the mutant residue based on the spatial coordinate sets of all heavy atoms of said each residue of the wild-type coronavirus spike protein and the inferred rotation angle,
wherein the step of estimating a candidate binding free energy value includes sub-steps of, for each residue in the wild-type coronavirus spike protein:
for a target interface between the mutant residue and a paired residue of the corresponding one of P number of antibodies,
for every two heavy atoms respectively of the mutant residue and the paired residue of the corresponding one of P number of antibodies, calculating a value of atomic-level energy and an Euclidean distance based on the spatial coordinate sets of the heavy atoms of the corresponding one of the P number of coronavirus spike protein-antibody complexes and the spatial coordinate sets of the heavy atoms of the mutant residue, and
calculating, based on the values of atomic-level energy and the Euclidean distances thus calculated, an atomic distance related to the target interface and an atomic interaction force of the target interface;
obtaining relevant information that is related to said each residue of the wild-type coronavirus spike protein and the mutant residue from amino acid physicochemical properties data that contains information related to physicochemical properties of amino acids; and
estimating the candidate binding free energy value of the target interface by feeding, into the pre-established model, the atomic distance related to the target interface, the atomic interaction force of the target interface and the relevant information.

7. A computing system for determining a target epitope on a specific virus so as to facilitate design of mutation-tolerable vaccine, the specific virus including a wild-type coronavirus spike protein having a plurality of residues, said computing system comprising:
a storage device configured to store a pre-established model;
an input module configured to receive sequence data of a plurality of strains of the specific virus, and to receive P number of entries of protein structure data that are respectively related to P number of coronavirus spike protein-antibody complexes, each of the P number of coronavirus spike protein-antibody complexes including the wild-type coronavirus spike protein and a corresponding one of P number of antibodies, P being a positive integer greater than one; and
a processor electrically connected to said storage device and said input module, and configured to
for an i^{th} one of the residues in the wild-type coronavirus spike protein, determine, based on the sequence data, a mutation frequency
$${F}_{i , j} = \frac{{M}_{i , j}}{N} ,$$
where i is a positive integer ranging from one to a total number of the residues of the wild-type coronavirus spike protein, j is a positive integer ranging from one to a total number of common amino acid residues other than the i^{th} one of the residues, N represents a total number of the strains of the specific virus, and M_{i,j} represents a total number of those of the strains of the specific virus in each of which the i^{th} one of the residues has mutated into a j^{th} one of the common amino acid residues,
analyze the P number of entries of protein structure data to obtain a plurality of interatomic distances respectively between a plurality of heavy-atom pairs, each of the heavy-atom pairs including two heavy atoms that respectively belong to the wild-type coronavirus spike protein and one of the P number of antibodies,
identify, based on the interatomic distances thus obtained, all contact residues in the P number of coronavirus spike protein-antibody complexes, wherein each of the contact residues is one of the residues in the wild-type coronavirus spike protein that includes an α-carbon which is spaced apart by a distance less than 5 Å from another α-carbon of a residue of one of the P number of antibodies that is paired with the contact residue,
for each residue in the wild-type coronavirus spike protein, count a total number of the contact residues Cᵢ that are related to said each residue and the P number of antibodies,
based on the P number of entries of protein structure data, for each target interface of each residue in the wild-type coronavirus spike protein, estimate a candidate binding free energy value of the target interface by using the pre-established model, each target interface being an interface between a mutant residue that is determined based on information related to properties of side-chain dihedral angles and bond rotation of amino acids, that possibly results from mutation of said each residue of the wild-type coronavirus spike protein and that is one of the common amino acid residues other than said each residue, and a paired residue of an antibody of one of the P number of coronavirus spike protein-antibody complexes that is paired with said each residue of the wild-type coronavirus spike protein,
for the condition that the i^{th} one of the residues in the wild-type coronavirus spike protein mutates into the j^{th} one of the common amino acid residues, select a greatest one of P number of candidate binding free energy values that are respectively related to the P number of coronavirus spike protein-antibody complexes as a representative binding free energy value B_{i,j},
for each of the representative binding free energy values, normalize the representative binding free energy value B_{i,j} into a normalized binding free energy value H_{i,j} by using min-max scaling in a manner that the normalized binding free energy value H_{i,j} ranges from zero to one,
for the condition that the i^{th} one of the residues in the wild-type coronavirus spike protein mutates into the j^{th} one of the common amino acid residues, determine a mutation effect score E_{i,j} based on the mutation frequency F_{i,j}, the total number of the contact residues Cᵢ and the normalized binding free energy value H_{i,j} in a manner that the mutation effect score E_{i,j} ranges from zero to one,
generate a mutation effect epitope map that is related to the mutation effect scores determined for all residues in the wild-type coronavirus spike protein and all of the common amino acid residues, and
determine, based on the mutation effect epitope map, a region in the wild-type coronavirus spike protein as the target epitope.

8. The computing system as claimed in claim 7, further comprising an output module electrically connected to said processor, and configured to be controlled by said processor to present the mutation effect epitope map and the target epitope.

9. The computing system as claimed in claim 7, wherein said processor is configured to normalize the representative binding free energy value B_{i,j} by calculating the normalized binding free energy value as
$${H}_{i , j} = \frac{{B}_{i , j} - min ( B )}{max ( B ) - min ( B )} ,$$
where max(B) represents a greatest one of all of the representative binding free energy values, and min(B) represents a smallest one of all of the representative binding free energy values.

10. The computing system as claimed in claim 7, wherein said processor is configured to determine the mutation effect score E_{i,j} by calculating the mutation effect score as
$${E}_{i , j} = \frac{{F}_{i , j} + \frac{{C}_{i}}{max ( C ) - min ( C )} + {H}_{i , j}}{3} ,$$
where max(C) represents a greatest one of all of the total numbers of the contact residues, and min(C) represents a smallest one of all of the total numbers of the contact residues, the total numbers of the contact residues corresponding respectively to the residues in the wild-type coronavirus spike protein.

11. The computing system as claimed in claim 7, wherein the pre-established model is implemented by a deep neural network (DNN), and is trained by using a plurality of training sets that respectively correspond to a plurality of training protein complexes, each of the training protein complexes including at least one pair of training residues that are respectively in two protein chains of the training protein complex and that are related to a training interaction interface; and each of the training sets contains, for each of the at least one pair of training residues included in the corresponding one of the training protein complexes, an atomic distance that is related to the training interaction interface, an atomic interaction force of the training interaction interface, a binding free energy value of the training interaction interface, and information related to physicochemical properties of amino acids that are related to the pair of training residues.

12. The computing system as claimed in claim 7, wherein:
said storage device is further configured to store amino acid structure data that contains information related to properties of backbone dihedral angles, side-chain dihedral angles and bond rotation of amino acids, and to store amino acid physicochemical properties data that contains information related to physicochemical properties of amino acids;
each of the P number of entries of protein structure data contains spatial coordinate sets respectively of all atoms of the corresponding one of the P number of coronavirus spike protein-antibody complexes;
said processor is further configured to, for each of the P number of entries of protein structure data,
from the entry of protein structure data, obtain spatial coordinate sets respectively of all heavy atoms of the corresponding one of the P number of coronavirus spike protein-antibody complexes,
for each residue in the wild-type coronavirus spike protein of the corresponding one of the P number of coronavirus spike protein-antibody complexes,
determine, based on information related to properties of side-chain dihedral angles and bond rotation of amino acids, a mutant residue that possibly results from mutation of said each residue of the wild-type coronavirus spike protein,
obtain an inferred rotation angle that is related to a side chain of said each residue of the wild-type coronavirus spike protein from the amino acid structure data, and
calculate spatial coordinate sets respectively of all heavy atoms of the mutant residue based on the spatial coordinate sets of all heavy atoms of said each residue of the wild-type coronavirus spike protein and the inferred rotation angle;
for each residue in the wild-type coronavirus spike protein, said processor is further configured to,
for a target interface between the mutant residue and a paired residue of the corresponding one of P number of antibodies,
for every two heavy atoms respectively of the mutant residue and the paired residue of the corresponding one of P number of antibodies, calculate a value of atomic-level energy and an Euclidean distance based on the spatial coordinate sets of the heavy atoms of the corresponding one of the P number of coronavirus spike protein-antibody complexes and the spatial coordinate sets of the heavy atoms of the mutant residue, and
calculate, based on the values of atomic-level energy and the Euclidean distances thus calculated, an atomic distance related to the target interface and an atomic interaction force of the target interface,
obtain relevant information that is related to said each residue of the wild-type coronavirus spike protein and the mutant residue from the amino acid physicochemical properties data, and
estimate the candidate binding free energy value of the target interface by feeding, into the pre-established model, the atomic distance related to the target interface, the atomic interaction force of the target interface and the relevant information.
